(19) United States
(12) Patent Application Publication
(10) Pub. No.: US 2024/0423556 A1
(43) Pub. Date: Dec. 26, 2024

(54) WAVEFORM DISPLAY APPARATUS, WAVEFORM DISPLAY SYSTEM, WAVEFORM DISPLAY METHOD, AND COMPUTER-READABLE MEDIUM

(71) Applicant: Otoichi NAKATA, Kanagawa (JP)

(72) Inventor: Otoichi NAKATA, Kanagawa (JP)

(21) Appl. No.: 18/750,548

(22) Filed: Jun. 21, 2024

(30) Foreign Application Priority Data
Jun. 23, 2023 (JP) 2023-103456

(51) Int. Cl. A61B 5/00; A61B 5/245; A61B 5/384

(52) U.S. Cl. CPC A61B 5/743; A61B 5/7264; A61B 5/245; A61B 5/384; A61B 5/4094

(57) ABSTRACT

A waveform display apparatus includes: a collateral information drawing unit configured to draw collateral information on waveforms with respect to the waveforms in accordance with a predetermined time, the waveforms being acquired by a plurality of sensors and displayed on a screen; a waveform extraction unit configured to extract a characteristic waveform from waveform data; a representative time determination unit configured to determine a representative time of the characteristic waveform extracted by the waveform extraction unit; and a waveform classification unit configured to classify the characteristic waveform extracted by the waveform extraction unit. The collateral information drawing unit is configured to display collateral information corresponding to the representative time of the characteristic waveform in accordance with a classification result by the waveform classification unit, the representative time being determined by the representative time determination unit.

### CROSS-REFERENCE TO RELATED APPLICATION

The present application claims priority under 35 U.S.C. § 119 to Japanese Patent Application No. 2023-103456, filed on Jun. 23, 2023. The contents of which are incorporated herein by reference in their entirety.

### BACKGROUND OF THE INVENTION

### 1. Field of the Invention

The present invention relates to a waveform display apparatus, a waveform display system, a waveform display method, and a computer-readable medium.

### 2. Description of the Related Art

In magneto-encephalography, dipole analysis is performed with respect to an origin of epilepsy in a brain based on a change in magnetic fields around the brain and a position at which the epilepsy has occurred is drawn on MRI, which contributes to identification of epileptogenesis.

In recent years, with the improvement of calculators and the development of artificial intelligence, attempts are being made to reduce a burden on an analyzer by extracting an interval that includes a characteristic waveform from long-time magneto-encephalography waveform data or a plurality of pieces of recorded magneto-encephalography waveform data, performing dipole analyses in a cross-sectional manner, and detecting candidates of an epileptic spike.

Japanese Unexamined Patent Application Publication No. 2021-069929 discloses a technique for determining accuracy of appearance of characteristic waveform information (Interictal Epileptiform Discharge (IED)) to determine a time at which the characteristic waveform information appears and extract a sensor, and displays the accuracy of appearance in color on a waveform.

However, according to the related technique, when the waveform of the candidate for the epileptic spike and a dipole extraction result are to be examined, there is a problem in that a reason for positive is not detailed and it is difficult to determine whether to adopt the result. Further, according to the related technique, information for detecting and correcting false negative that needs to be extracted among a large number of pieces of negative is not adequately provided, and therefore, there is a problem in that a user may miss the false negative.

### SUMMARY OF THE INVENTION

According to an aspect of the present invention, a waveform display apparatus includes a collateral information drawing unit, a waveform extraction unit, a representative time determination unit, and a waveform classification unit. The collateral information drawing unit is configured to draw collateral information on waveforms with respect to the waveforms in accordance with a predetermined time, the waveforms being acquired by a plurality of sensors and displayed on a screen. The waveform extraction unit is configured to extract a characteristic waveform from waveform data. The representative time determination unit is configured to determine a representative time of the characteristic waveform extracted by the waveform extraction unit. The waveform classification unit is configured to classify the characteristic waveform extracted by the waveform extraction unit. The collateral information drawing unit is configured to display collateral information corresponding to the representative time of the characteristic waveform in accordance with a classification result by the waveform classification unit, the representative time being determined by the representative time determination unit.

### BRIEF DESCRIPTION OF THE DRAWINGS

FIG. 1 is a schematic diagram illustrating a configuration of a biological signal measurement system according to a first embodiment;

FIG. 2 is a block diagram schematically illustrating a functional configuration of a server;

FIG. 3 is a diagram illustrating an example of a hardware configuration of an information processing apparatus;

FIG. 4 is a diagram illustrating an example of a functional block configuration of the information processing apparatus;

FIG. 5 is a flowchart illustrating the flow of a dipole estimation process;

FIGS. 6A and 6B are diagrams illustrating an example of calculation of an IED probability map;

FIG. 7 is a diagram illustrating an example of waveform display;

FIG. 8 is a diagram illustrating examples of waveform display cursors;

FIG. 9 is a diagram illustrating an example in which waveform additional information is displayed in a superimposed manner;

FIG. 10 is a diagram illustrating an example of layers of waveform superimposed display;

FIG. 11 is a diagram illustrating another example of the layers of the waveform superimposed display;

FIG. 12 is a diagram illustrating an example of selection of layers to be displayed;

FIG. 13 is a diagram illustrating another example in which the waveform additional information is displayed in a superimposed manner;

FIG. 14 is a diagram illustrating an example of a setting screen of a dipole extraction condition;

FIG. 15 is a diagram illustrating an example of display for correcting a result;

FIGS. 16A and 16B are diagrams illustrating examples of display of annotation;

FIGS. 17A and 17B are diagrams illustrating examples of display of a dipole list;

FIG. 18 is a diagram illustrating a modification of the annotation and the dipole list;

FIG. 19 is a flowchart illustrating the flow of a series of operation; and

FIG. 20 is a flowchart illustrating the flow of a training process for generating an IED probability map by using machine learning according to a second embodiment.

The accompanying drawings are intended to depict exemplary embodiments of the present invention and should not be interpreted to limit the scope thereof. Identical or similar reference numerals designate identical or similar components throughout the various drawings.

### DESCRIPTION OF THE EMBODIMENTS

The terminology used herein is for the purpose of describing particular embodiments only and is not intended to be limiting of the present invention.

As used herein, the singular forms “a”, “an” and “the” are intended to include the plural forms as well, unless the context clearly indicates otherwise.

In describing preferred embodiments illustrated in the drawings, specific terminology may be employed for the sake of clarity. However, the disclosure of this patent specification is not intended to be limited to the specific terminology so selected, and it is to be understood that each specific element includes all technical equivalents that have the same function, operate in a similar manner, and achieve a similar result.

An embodiment of the present invention will be described in detail below with reference to the drawings.

An embodiment has an object to display required information in a visible manner and provide information that allows a user for easy recognition.

Embodiments of a waveform display apparatus, a waveform display system, a waveform display method, and a non-transitory computer readable recording medium having stored therein a program will be described in detail below with reference to the accompanying drawings.

The present invention is not limited by the embodiments below, and constituent elements in the embodiments described below include one that can be easily thought of by a person skilled in the art, one that is practically identical, and one that is within an equivalent range. Further, within the scope not departing from the gist of the embodiments described below, various omission, replacement, and modifications of the constituent elements may be made.

The present embodiment has a feature as described below in terms of calculation of a probability map of an Interictal Epileptiform Discharge (IED). Specifically, with use of the IED probability map, it is possible to detect an onset time of the IED that is required for an equivalent current dipole analysis method and select a sensor, which is characteristic.

When a doctor performs analysis, it is ideal to analyze an onset site of an IED by the equivalent current dipole method; however, it is known that an signal-to-noise ratio (S/N) of the onset site is low and, in reality, a time of the IED is determined in a range from the onset site to a peak of the IED. In the present embodiment, analysis is performed without limiting the analysis to the onset site.

### First Embodiment

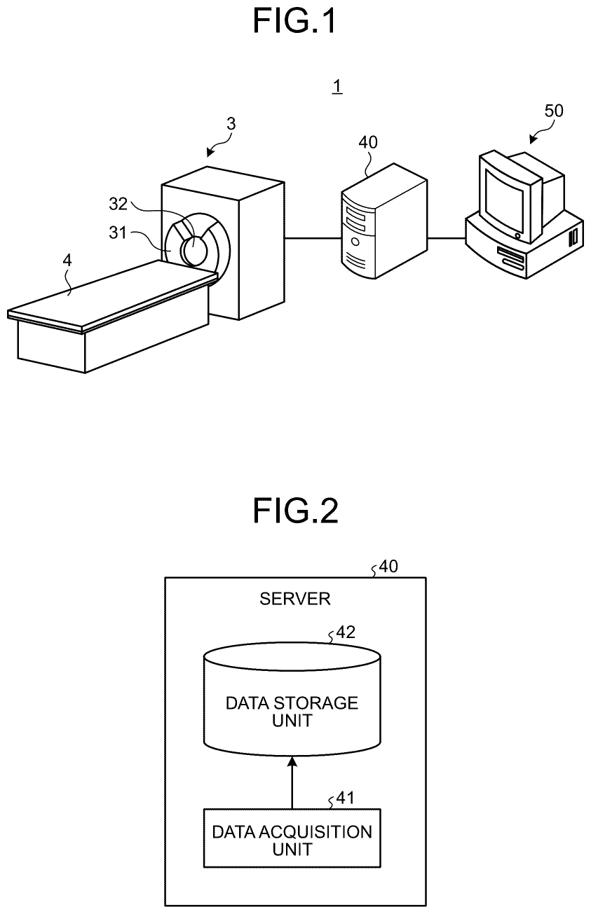

Overview of biological signal measurement system FIG. 1 is a schematic diagram illustrating a configuration of a biological signal measurement system according to a first embodiment. An overview of a biological signal measurement system 1 according to the present embodiment will be described below with reference to FIG. 1.

The biological signal measurement system 1 is a waveform display system that measures a plurality of kinds of biological signals (for example, a Magneto-encephalography (MEG) signal and an Electro-encephalography (EEG) signal) on a subject from a specific emission source (living body site), and displays the biological signals. Meanwhile, in the present invention, the biological signals to be measured are not limited to the magneto-encephalography signal and the electro-encephalography signal, but may be, for example, an electrical signal that is generated in response to heart activities (electrical signal that can be represented by an electro-cardiogram) or the like.

As illustrated in FIG. 1, the biological signal measurement system 1 includes a measurement apparatus 3 that measures one or more biological signals on a subject, a server 40 that records one or more kinds of biological signals that are measured by the measurement apparatus 3, and an information processing apparatus 50 that is a waveform display apparatus that analyzes one or more kinds of biological signals that are recorded in the server 40. The measurement apparatus 3 is, for example, a magneto-encephalography measurement apparatus that collects measurement values of a brain's magnetic field, a timing at which a stimulus is applied, or the like. Meanwhile, in FIG. 1, the server 40 and the information processing apparatus 50 are separately illustrated, but, for example, it is possible to adopt a configuration in which at least a part of functions included in the server 40 is incorporated in the information processing apparatus 50.

In the example illustrated in FIG. 1, a subject (to-be-measured person) lies down on a measurement table 4 with face up while electrodes serving as sensors for electro-encephalography are mounted on the head of the subject, and a head portion is inserted in a hollow 32 of a dewar 31 of the measurement apparatus 3. The dewar 31 is a holding container in an extremely low temperature environment using liquid helium, and a large number of magnetic sensors for magneto-encephalography are arranged inside the hollow 32 of the dewar 31. The measurement apparatus 3 collects electro-encephalography signals from the electrodes and magneto-encephalography signals from the magnetic sensors, and outputs data (hereinafter, may be referred to as “measurement data”) that includes the collected electro-encephalography signals and the collected magneto-encephalography signals to the server 40. The measurement data that is output to the server 40 is read, displayed, and analyzed by the information processing apparatus 50. In general, the dewar 31 in which the magnetic sensors are incorporated and the measurement table 4 are arranged in a magnetic shielding room, but illustration of the magnetic shielding room is omitted in FIG. 1 for the sake of convenience.

The information processing apparatus 50 is an apparatus that displays waveform data of the magneto-encephalography signals that are obtained from the plurality of magnetic sensors and waveform data of the electro-encephalography signals that are obtained from the plurality of electrodes, in a synchronous manner on the same time axis. The electro-encephalography signal is a signal that represents electrical activity of a nerve cell (ion charge flow that occurs in dendrites of a neuron at the time of synaptic transmission) as a voltage value between the electrodes. The magneto-encephalography signal is a signal that represents minute magnetic field variation that occurs due to electrical activity of a brain. The brain's magnetic field is detected by a high-sensitive Superconducting Quantum Interference Device (SQUID) sensor. The electro-encephalography signal and the magneto-encephalography signal are examples of a “biological signal”.

FIG. 2 is a block diagram schematically illustrating a functional configuration of the server 40. As illustrated in FIG. 2, the server 40 includes a data acquisition unit 41 and a data storage unit 42.

The data acquisition unit 41 periodically acquires measurement data from the measurement apparatus 3. Here, the measurement data is each piece of waveform data that is measured by each of the magnetic sensors of the dewar 31 of the measurement apparatus 3.

The data storage unit 42 stores therein the measurement data that is acquired from the measurement apparatus 3.

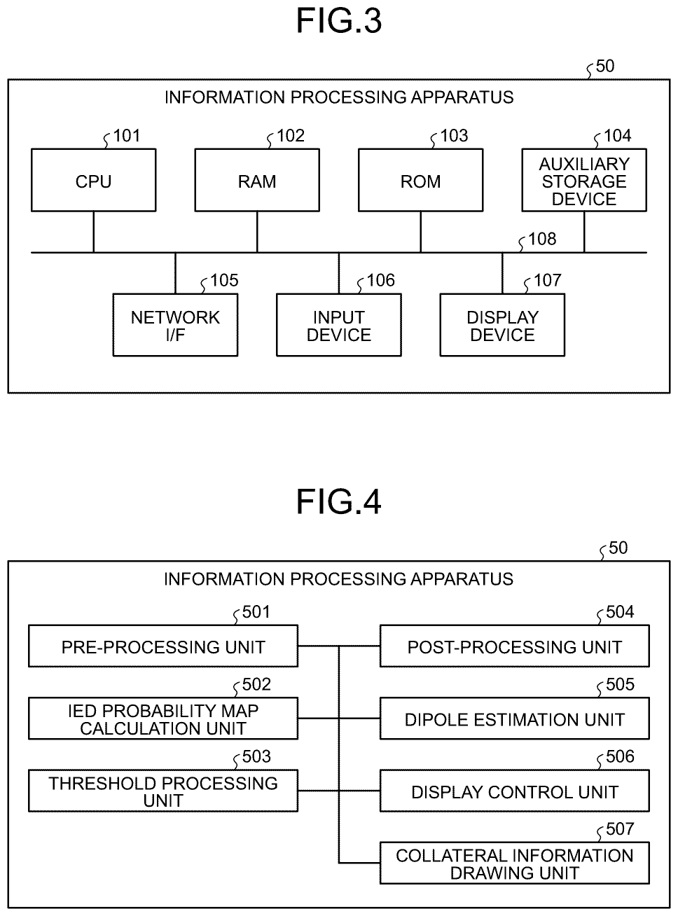

Hardware configuration of information processing apparatus FIG. 3 is a diagram illustrating an example of a hardware configuration of the information processing apparatus 50. The hardware configuration of the information processing apparatus 50 will be described below with reference to FIG. 3.

As illustrated in FIG. 3, the information processing apparatus 50 includes a Central Processing Unit (CPU) 101, a Random Access Memory (RAN) 102, a Read Only Memory (ROM) 103, an auxiliary storage device 104, a network interface (I/F) 105, an input device 106, and a display device 107, all of which are connected to one another via a bus 108.

The CPU 101 is an arithmetic device that controls entire operation of the information processing apparatus 50 and performs various kinds of information processing. The CPU 101 executes an information display program that is stored in the ROM 103 or the auxiliary storage device 104 and controls operation of displaying a measurement collection screen and an analysis screen.

The RAM 102 is a volatile storage device that is used as a work area of the CPU 101 and that stores therein main control parameters and information. The ROM 103 is a non-volatile storage device that stores therein a basic input-output program or the like. For example, it may be possible to store the information display program as described above in the ROM 103.

The auxiliary storage device 104 is a storage device, such as a Hard Disk Drive (HDD) or a Solid State Drive (SSD). The auxiliary storage device 104 stores therein, for example, a control program for controlling operation of the information processing apparatus 50, various kinds of data and files that are required for the operation of the information processing apparatus 50, and the like.

The network I/F 105 is a communication interface for performing communication with an apparatus, such as the server 40, on a network. The network I/F 105 is implemented by, for example, a Network Interface Card (NIC) or the like that is compliant with Transmission Control Protocol/Internet Protocol (TCP/IP).

The input device 106 is an input function of a touch panel, a keyboard, a mouse, a user interface, such as an operation button, or the like. The display device 107 is a display device that displays various kinds of information. The display device 107 is implemented by, for example, a display function of a touch panel, a Liquid Crystal Display (LCD), an organic Electro-Luminescence (EL), or the like. The display device 107 displays the measurement collection screen and the analysis screen, and updates the screen in accordance with input-output operation that is performed via the input device 106.

Meanwhile, the hardware configuration of the information processing apparatus 50 illustrated in FIG. 3 is one example, and it may be possible to add a different device. Further, the information processing apparatus 50 illustrated in FIG. 3 has the hardware configuration that is based on the assumption that the information processing apparatus 50 is a Personal Computer (PC) for example, but embodiments are not limited to this example, and it may be possible to adopt a mobile terminal, such as a tablet. In this case, the network I/F 105 may be any communication interface as long as the communication interface has a wireless communication function.

Functional block configuration of information processing apparatus FIG. 4 is a diagram illustrating an example of a functional block configuration of the information processing apparatus 50. The functional block configuration of the information processing apparatus 50 will be described below with reference to FIG. 4.

As illustrated in FIG. 4, the information processing apparatus 50 includes a pre-processing unit 501, an IED probability map calculation unit 502, a threshold processing unit 503, a post-processing unit 504, and a dipole estimation unit 505. In addition, the information processing apparatus 50 further includes a display control unit 506 and a collateral information drawing unit 507.

The pre-processing unit 501 performs pre-processing, such as extraction and augmentation of a sensor, down-sampling, application of a frequency filter, elimination of artifacts, a defective channel process, extraction of a time window, and standardization of magnetic field data.

As for the extraction and augmentation of a sensor, when an IED probability map (to be described later) is to be calculated, it may be possible to adopt a calculation method using all of sensors or it may be possible to perform calculation by using only a group of a fixed number of sensors that are grouped in advance. As for grouping of sensors, it may be possible to form a group in accordance with an anatomical criterion, such as a temporal lobe or a frontal lobe, or it may be possible to simply form a group of an arbitrary number of sensors that are located close to each other. Further, in a training process (to be described later), if the number of sensors at the time of calculation is smaller than the number of used sensors, it may be possible to expand fictional sensors.

The down-sampling is applied so as to adjust to a sampling frequency that is used at the time of training. Similarly, the same frequency filter that is applied to filtering at the time of training is applied to a frequency filter. The filter that is frequently used is a low-pass filter at 35 Hz, a band-pass filter at 3 Hz to 35 Hz, or the like.

As for the elimination of artifacts, to eliminate cardiac artifacts or artifacts due to blink or body motion, ICA (see E. Javier, H. Roberto, A. Daniel, F. Alberto, and L. C. Miguel, “Artifact removal in magnetoencephalogram background activity with independent component analysis”, IEEE Trans Biomed Eng, vol. 54, no. 11, pp. 1965-1973, 2007.), DSP (see K. Sekihara, Y. Kawabata, S. Ushio, S. Sumiya, S. Kawabata, Y. Adachi, and S. S. Nagarajan, “Dual signal subspace projection (DSP): a novel algorithm for removing large interference in biomagnetic measurements”, Journal of Neural Engineering, vol. 13, no. 3, p. 036007, 2016.), or the like may be applied.

The defective channel process indicates a process of eliminating a sensor for which a magnetic field change that exceeds a threshold that is set in advance is observed, or a process for performing interpolation using peripheral sensor values.

As for the extraction of a time window, it may be possible to adopt a method of shift by a length corresponding to the time window without overlap, a method of overlapping a half of the length corresponding to the time window, a method of overlapping one-fourth of the length corresponding to the time window, or the like. In the case of overlap, an additive average is performed for the overlapping portion at the time of calculation of the IED probability map (to be described later).

As for the standardization of magnetic field data, standardization is applied such that an average in the extracted time window is 0 and dispersion is 1. It may be possible to use a normalization method such that a range of the magnetic field set in advance is −1 to 1, in addition to the standardization.

The IED probability map calculation unit 502 functions as a waveform extraction unit that extracts a characteristic waveform (IED) from waveform data. The IED probability map calculation unit 502 calculates a probability map of the characteristic waveform. Further, the IED probability map calculation unit 502 compares each piece of waveform data acquired by a plurality of sensors with at least one or more pieces of characteristic waveform information.

The IED indicates a waveform, such as a Spike Wave, a Spike and Wave, a Poly Spike and Wave, or a Sharp Wave, which is a characteristic of an epilepsy lesion site.

The threshold processing unit 503 narrows down, from the IED probability map that is obtained by the IED probability map calculation unit 502, a time and a region of sensors for which an IED probability is high, by using a threshold. The threshold processing unit 503 functions as a waveform classification unit that classifies the characteristic waveform that is extracted by the IED probability map calculation unit 502. The threshold processing unit 503 determines a class of the characteristic waveform in accordance with appearance accuracy information that corresponds to a time and a subject sensor identified by the post-processing unit 504.

The post-processing unit 504 performs post-processing for extracting a sampling time point and a sensor used for dipole estimation from the IED probability map that is subjected to a threshold process by the threshold processing unit 503. Further, if a plurality peaks are present in a peak detection method or a map (a case in which two or more lesion sites are present), the post-processing unit 504 performs post-processing for separating the map. The post-processing unit 504 functions as a representative time determination unit that determines a representative time of the waveform that is extracted by the IED probability map calculation unit 502. The post-processing unit 504 determines appearance accuracy of the characteristic waveform information in at least a fixed interval of the waveform data, based on a correlation ratio between a peak interval of the waveform data and the characteristic waveform information. Further, the post-processing unit 504 identifies a time at which an interval that matches the characteristic waveform information appears and a subject sensor, based on the appearance accuracy.

The dipole estimation unit 505 performs dipole estimation by using the sensor and the sampling time point of the onset site of the IED that are obtained by a process prior to the dipole estimation unit 505 (see M. Scherg, “Fundamentals of dipole source potential analysis”, in Auditory Evoked Magnetic Fields and Potentials, M. Hoke, F. Grandori, and G. L. Romani, Eds. Basel, Switzerland: Karger, 1989, vol. 6).

A flow of a dipole estimation process performed by the dipole estimation unit 505 will be described below.

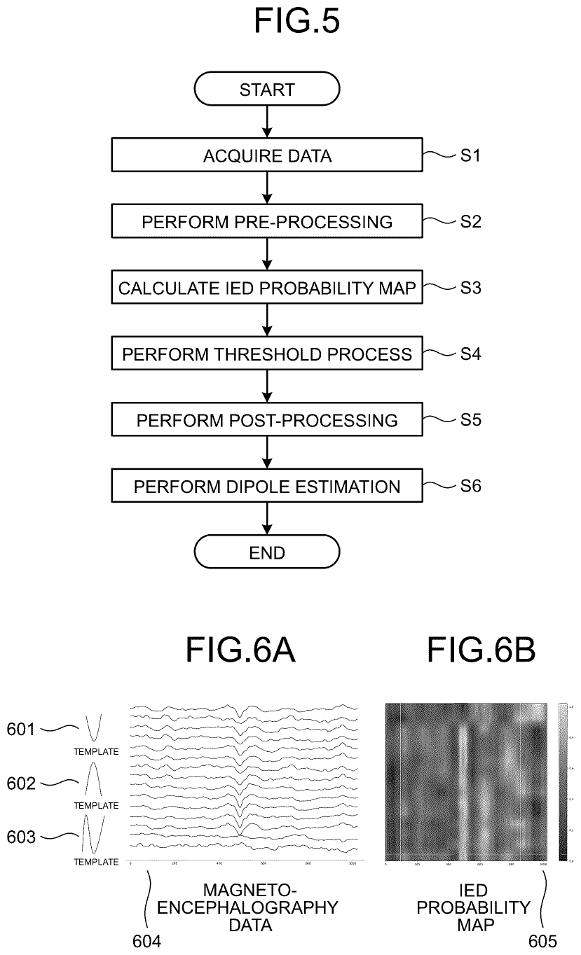

FIG. 5 is a flowchart illustrating the flow of the dipole estimation process. Before the process, the measurement apparatus 3 performs magneto-encephalography measurement, and outputs individual waveform data that is measured by each of the magnetic sensors of the dewar 31 to the server 40. The server 40 stores the individual waveform data that is measured by each of the magnetic sensors of the dewar 31 of the measurement apparatus 3 in the data storage unit 42.

As illustrated in FIG. 5, first, the pre-processing unit 501 acquires the individual waveform data that is measured by each of the magnetic sensors of the dewar 31 of the measurement apparatus 3 from the data storage unit 42 of the server 40 (Step S1).

Subsequently, the pre-processing unit 501 performs pre-processing, such as extraction and augmentation of a sensor, down-sampling, application of a frequency filter, elimination of artifacts, a defective channel process, extraction of a time window, and standardization of magnetic field data, on the acquired data (Step S2).

Then, the IED probability map calculation unit 502 calculates a probability map of the characteristic waveform (IED) (Step S3).

Subsequently, the threshold processing unit 503 performs a threshold process on the IED probability map that is acquired by the IED probability map calculation unit 502 (Step S4). Specifically, the threshold processing unit 503 narrows down, by using a predetermined threshold, a time and a region of sensors for which the IED probability is high from the IED probability map that is obtained by the IED probability map calculation unit 502.

Meanwhile, in the threshold processing unit 503, it is possible to select only waveforms that are more likely to be IEDs with an increase in the threshold; however, in this case, the number of IEDs used for dipole estimation is reduced. In contrast, in the threshold processing unit 503, if the threshold is reduced, it is possible to detect an increased number of IEDs although erroneous detection increases. Further, magneto-encephalography data is largely affected by individual differences, and therefore, if the threshold is not reduced, there may be a case in which IED can hardly be detected.

Therefore, the threshold processing unit 503 may be configured to interactively control the number of IEDs to be detected in cooperation with a User Interface (UI).

The threshold processing unit 503 basically uses a preset value, such as “0.8”, as the threshold.

Then, the post-processing unit 504 performs post-processing for extracting a sampling time point and a sensor that are used for dipole estimation from the IED probability map that is subjected to the threshold process by the threshold processing unit 503 (Step S5).

Specifically, if the threshold processing unit 503 is applied, a time and a region of sensors with values equal to or larger than a predetermined probability are extracted. To clarify a time point of peak detection or IED detection, it may be possible to apply a certain filter, such as a Gaussian filter, on the extracted region of sensors before peak detection. By detecting a peak after applying a certain filter, such as a Gaussian filter, to the extracted region of sensors, it is possible to determine the time point of the IED. When detecting a peak, it may be possible to adopt a one-dimensional probability map in which the probability map including the extracted region of sensors is subjected to additive average in a sensor direction. When additive average is not performed, it may be possible to detect a peak for each of the sensors and determine the time of the IED by obtaining an average of peak time points.

The threshold processing unit 503 extracts sensors that have values equal to or larger than a predetermined probability at the detected peak time point, as sensors that are used for dipole estimation. In this case, it is known that, if the number of sensors used for dipole estimation is reduced, stability of a dipole estimated solution is reduced. Therefore, in this example, when the number of the selected sensors is smaller than a predetermined value, it may be possible not to perform dipole estimation at this time point of the IED.

Furthermore, as another sensor extraction method, it may be possible to define a group of sensors in advance and expand an extracted sensor to all of the sensors in a group to which the extracted sensor belongs, so that it is possible to improve the stability of the dipole estimated solution. Here, it is assumed that the group is basically set in accordance with an anatomical criterion, such as near a temporal lobe or near a frontal lobe; however, it may be possible to simply adopt a neighboring region including an arbitrary number of sensors as a single group.

Lastly, the dipole estimation unit 505 performs dipole estimation by using the sensors and the sampling time point of the onset site of the IED that are obtained by a process prior to the dipole estimation unit 505 (see M. Scherg, “Fundamentals of dipole source potential analysis”, in Auditory Evoked Magnetic Fields and Potentials, M. Hoke, F. Grandori, and G. L. Romani, Eds. Basel, Switzerland: Karger, 1989, vol. 6) (Step S6). In this example, it is possible to adopt, other than the dipole estimation, a current source analysis method using a spatial filtering method, such as the minimum norm method (see K. Sekihara, M. Sahani, and S. S. Nagarajan, “Localization bias and spatial resolution of adaptive and non-adaptive spatial filters for MEG source reconstruction”, NeuroImage, vol. 25, no. 4, pp. 1056-1067, 2005.) or the LCMV Beamformer method (see B. V. Veen, W. V. Drongelen, M. Yuchtman, and A. Suzuki, “Localization of brain electrical activity via linearly constrained minimum variance spatial filtering”, IEEE Transactions on Biomedical Engineering, vol. 44, no. 9, pp. 867-880, 1997.). The spatial filtering method is a method in which approximately tens of thousands of dipoles are arranged in a brain in advance, and it is possible to obtain a temporal change of an electric current in each of the dipoles.

FIGS. 6A and 6b are diagrams illustrating an example of calculation of an IED probability map. In the example illustrated in FIGS. 6A and 6B, the IED probability map calculation unit 502 uses template matching. The IED probability map calculation unit 502 detects the IED by using a template in which a shape of the IED is generalized and which is generated based on the literature that investigates a morphological characteristic of the IED in magneto-encephalography (see R. Nowak, M. Santiuste, and A. Russi, “Toward a definition of MEG spike: Parametric description of spikes recorded simultaneously by MEG and depth electrodes”, Seizure, vol. 18, no. 9, pp. 652-655, 2009.).

As illustrated in FIG. 6A, the IED probability map calculation unit 502 scans templates, such as templates 601 to 603, chronologically over magneto-encephalography data of a single sensor in magneto-encephalography data 604, and, as illustrated in FIG. 6B, generates an IED probability map 605 that is a two-dimensional map based on similarities. The map generated at this time is not a probability map in a strict sense; however, the map can be used as the map for the purpose, and therefore is referred to as the probability map for the sake of convenience.

The similarities can be calculated by using a technique, such as Normalized Cross-Correlation (NCC) or Zero-mean Normalized Cross-Correlation (ZNCC), in which a similarity calculation method for template matching in an image is one-dimensionalized, a technique in which convolution with a filter is simply normalized, or the like. As for detection of the IED, it may be possible to apply some different methods, such as a derivation method based on detection of steep transition from a base line of the IED, instead of the template matching.

Meanwhile, when the IED probability map calculation unit 502 does not use machine learning, dipole estimation at the peak position may lead to an unfavorable result, and therefore, it is required to perform post-processing such that, for example, dipole estimation is performed while adopting, as the sampling time point, a certain time point that is slightly shifted forward from the peak position on a time axis.

With use of an automatic IED search method using the algorithm as described above, it is possible to automate search of the IED and determination of the onset site.

The display control unit 506 is configured to display waveforms that are acquired by the plurality of sensors on a display screen in a side-by-side manner.

The collateral information drawing unit 507 draws collateral information of the waveform in accordance with a predetermined time. The collateral information drawing unit 507 displays collateral information corresponding to a waveform representative time that is determined by the post-processing unit 504, in accordance with a waveform classification result that is obtained by the threshold processing unit 503.

For example, the collateral information drawing unit 507 draws the number of subject sensors that correspond to a time that is determined as the collateral information. Further, the collateral information drawing unit 507 provides highlighted display in accordance with the number of subject sensors that correspond to the time that is determined as the collateral information.

An example of a waveform display method in the display control unit 506 will be described below.

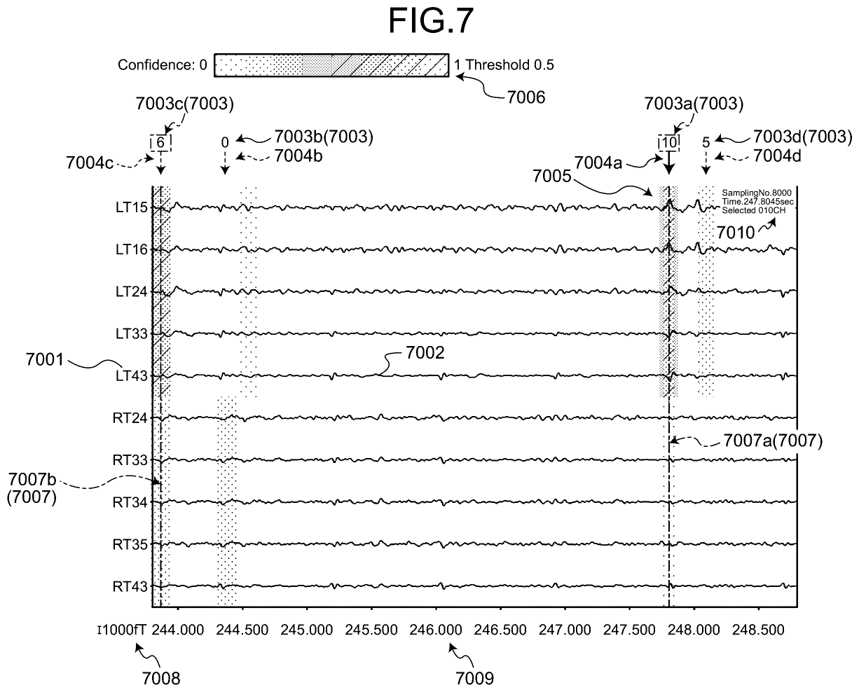

FIG. 7 is a diagram illustrating an example of waveform display. In the example of the waveform display illustrated in FIG. 7, the display control unit 506 displays label names 7001 of measured sensors, measured waveforms 7002 that correspond to the label names 7001, and a sensor number 7073 indicating the number of sensors that exceed a predetermined probability threshold in the IED probability map. Meanwhile, when dipole estimation results are obtained, the sensor number 7003 as the number of sensors that exceed the predetermined probability threshold indicates the number of sensors at the time of the dipole estimation.

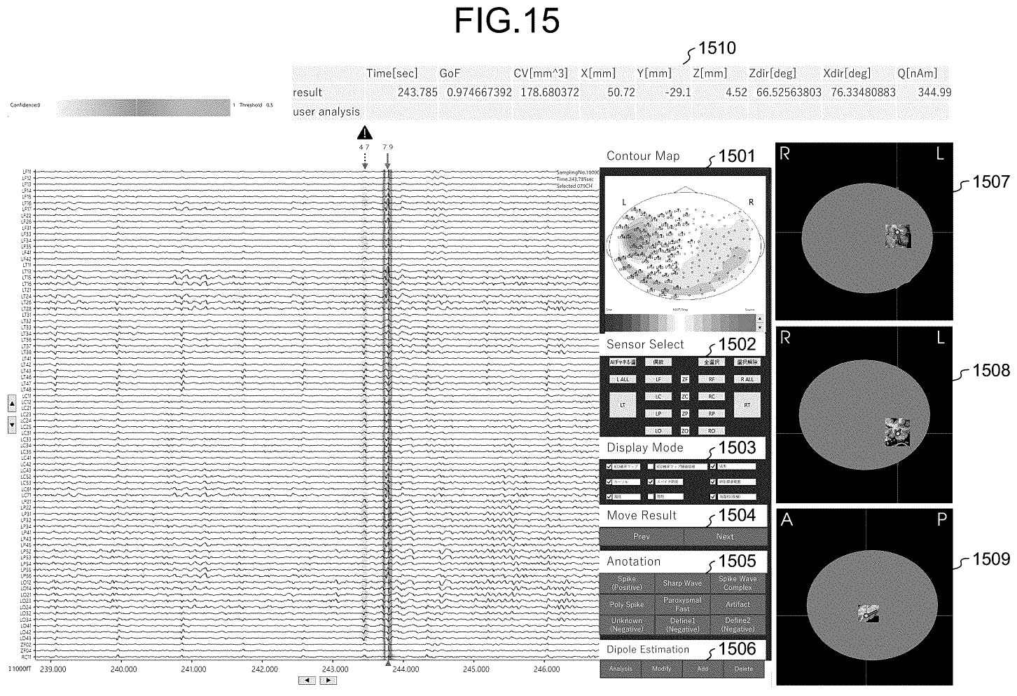

A sensor number 7003a is a display example of annotation for the case where a detection result is positive. Display of a chain line indicates an example of highlighted display that is adopted to annotation that is likely to be false positive. Meanwhile, the display is not limited to the chain line, and any method is applicable as long as the method makes it possible to distinguish and highlight between false positive and positive. For example, it is possible to adopt a method of changing a display color of a value, a method of setting an exclamation mark “!” above the value, or the like. Further, in a display example as illustrated in FIG. 15 (to be described later), it may be possible to write a specific name when an Annotation 1505 (see FIG. 15) is set. Furthermore, it may be possible to combine the above-described methods. Meanwhile, an example of a criterion to determine the possibility of false positive will be described later.

A sensor number 7003b is a display example of annotation for the case where a detection result is negative. For example, if there is at least one sensor for which the IED probability at an indicated time is larger than 0, it is indicated that the number of sensors that exceed the threshold after the threshold process with respect to the IED probability is zero. Meanwhile, in the case of negative that does not correspond to a sensor number 7003c (to be described later), that is, in the case of like the sensor number 7003, it may be possible to adopt a simple display mode by omitting display of numerals. In this case, it is preferable to adopt a hidden mode because it is less required for a user to check details.

The sensor number 7003c is a display example of annotation for the case where a detection result is negative and is detected as a false negative candidate. Similarly to the case of false positive, this is an example of a display method of highlighted display that is adopted to annotation that is likely to be false negative. In the example illustrated in FIG. 7, display of a chain line indicates an example of highlighted display that is adopted to annotation that is likely to be false negative. Meanwhile, it is possible to adopt the same highlighting method as the sensor number 7003a, or it is possible to adopt a different display method to distinguish from the sensor number 7003a. Meanwhile, a criterion to determine the possibility of false negative will be described later.

A sensor number 7003d is a display example for the case where a detection result is negative. Here, an example is illustrated in which it is determined as negative because the number of sensors for which probability values in the IED probability map exceed a threshold is smaller than a predetermined value. Even in this example, annotation that corresponds to false negative is displayed in a highlighted manner, similarly to the sensor number 7003c. This example includes a case in which, for example, it is determined as negative because the IED probability is high and the number of selected sensors does not reach a predetermined number.

In the example of the waveform display illustrated in FIG. 7, a time 7004 of positive or negative is displayed. The negative includes only those for which probability values in the IED probability map are larger than zero and final results are determined as negative, and those for which probability values in the IED probability map are zero are excluded from display targets.

A time 7004a is display that indicates a representative time of the positive. In FIG. 7, a solid arrow indicates the representative time of the positive so as to be distinguished from a dotted arrow indicating the negative as will be described later. Meanwhile, it may be possible to adopt a different display method for distinguishing between the negative and the positive, such as a method of changing colors or shapes of the arrows, rather than using the solid line and the dotted line. This example indicates that it is determined as positive after dipole estimation is performed. A dipole estimation time 7007a is indicated by a chain line.

A time 7004b is display that indicates a representative time of the negative. In FIG. 7, the dotted arrow indicates the representative time of the negative.

A time 7004c is display that indicates a representative time of the negative. In FIG. 7, a dotted arrow indicates the representative time of the negative. This example indicates that it is determined as negative after dipole estimation is performed. A dipole estimation time 7007b is indicated by a chain line.

A time 7004d is display that indicates a representative time of the negative. In FIG. 7, a dotted arrow indicates the representative time of the negative. Meanwhile, dipole estimation is not performed and the chain line that represents any of dipole estimation times 7007 is not displayed.

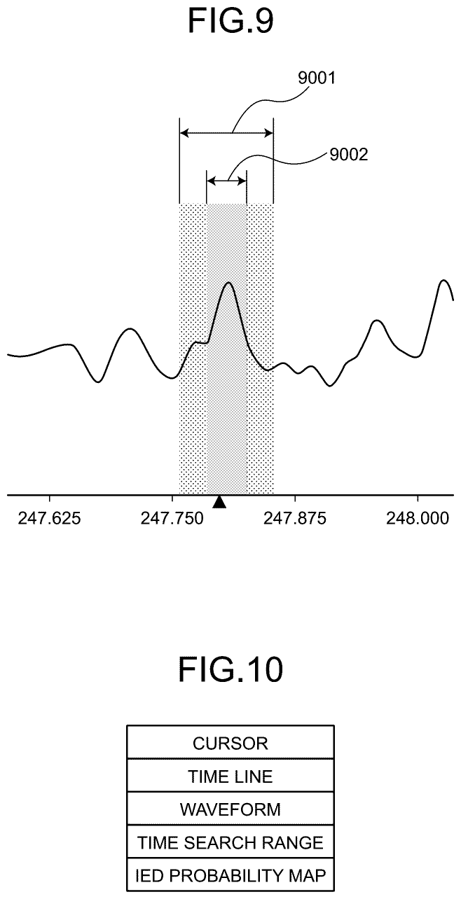

In the example of the waveform display illustrated in FIG. 7, the display control unit 506 displays an IED probability map 7005 in a background of the waveforms 7002. The IED probability map 7005 is an IED probability map that corresponds to each of regions of displayed sensors that are arranged at equal intervals in an entire waveform drawing region. In the example of the waveform display illustrated in FIG. 7, the inside of a rectangle including lines drawing a vertical axis line and a lateral axis line serves as the drawing region, and a vertical direction is divided into 10 regions corresponding to the number of selected sensors. In this example, only the IED probability map is displayed; however, it may be possible to simultaneously draw search information on a dipole estimation time posterior to the IED probability map as in an example illustrated in FIG. 9 (to be described later).

In the example of the waveform display illustrated in FIG. 7, the display control unit 506 displays a shading map 7006 that indicates a meaning of shading of the IED probability map. As the example of the shading map 7006, an IED probability threshold (Threshold) is represented by a decimal number between 0 and 1, which corresponds to a probability between 0 and 100%. A threshold for the IED probability after the threshold process corresponds to a Threshold value (0.5 (50%)). A dotted line in the center of the shading map 7006 is a corresponding position of the threshold on the shading map.

In the example of the waveform display illustrated in FIG. 7, the display control unit 506 displays the dipole estimation times 7007 (7007a and 7007b), at which dipole estimation is performed, on the detected characteristic waveforms. In the example of the waveform display in FIG. 7, the dipole estimation times 7007 (7007a and 7007b) are displayed by using the chain lines. Meanwhile, the dipole estimation times 7007 (7007a and 7007b) may be displayed by using different line styles depending on classification types (positive, negative, false negative, and false positive) so as to prevent erroneous recognition, or by using different formats, such as different line colors, depending on groups that are defined by a user, such as types of annotation of the Annotation 1505 (see FIG. 15), for example.

In the example of the waveform display illustrated in FIG. 7, the display control unit 506 displays a display unit/scale 7008 that represents measured values on the vertical axis, a representative scale 7009 that represents measurement times on the lateral axis, and a waveform information display portion 7010 that indicates a sampling number, a time, the number of displayed waveforms, and the like at a focus point on a waveform.

A display example of a waveform display cursor in the display control unit 506 will be described below.

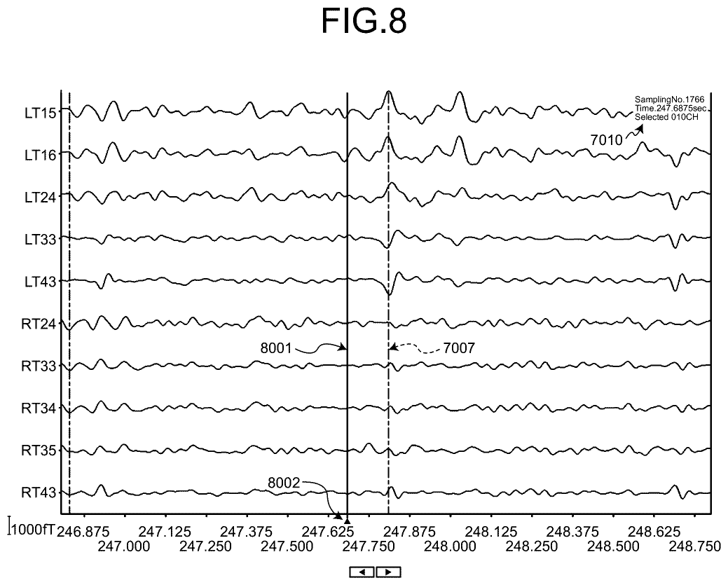

FIG. 8 is a diagram illustrating examples of waveform display cursors. A waveform display cursor 8001 illustrated in FIG. 8 is a cursor that indicates a waveform focus time. The waveform display cursor 8001 can be displayed in a transparent manner by solid line display so as to be simultaneously displayed even when the waveform display cursor 8001 overlaps with the dipole estimation time 7007. Meanwhile, the waveform display cursor 8001 can be operated at an arbitrary time by input via a keyboard or a mouse.

Further, a waveform display cursor 8002 illustrated in FIG. 8 is also a cursor that indicates the waveform focus time. The waveform display cursor 8002 is arranged so as to maintain visibility when the dipole estimation time 7007 and information overlap with each other. The waveform display cursor 8002 may be omitted depending on a display mode.

As illustrated in FIG. 8, the display control unit 506 displays, in the waveform information display portion 7010, a time that corresponds to cursor positions of the waveform display cursors 8001 and 8002.

An example of waveform additional information that is collateral information of the waveform to be drawn by the collateral information drawing unit 507 in accordance with a predetermined time will be described below.

FIG. 9 is a diagram illustrating an example in which the waveform additional information is displayed in a superimposed manner. Waveform additional information 9001 illustrated in FIG. 9 is an example of a display range of the IED probability map. The waveform additional information 9001 indicates a temporal range for providing a characteristic waveform.

Further, waveform additional information 9002 is an example of display of an area corresponding to a time width that is retrieved as a candidate dipole estimation time in the display range of the IED probability map.

As for the dipole estimation time, a time and a sensor are selected from the IED probability map. In the dipole estimation, one that meets a predetermined criterion by using a statistical index (for example, Good of Fitness, Confidence Volume, moment (Intensity), or the like) is adopted as a dipole, and one that does not meet the predetermined criterion is not adopted as a dipole. In other words, it may be determined as negative in some cases. In contrast with negative determination as described above, it may be possible to adopt a method of performing dipole estimation in a cross-sectional manner at times before and after a subject time, adopting a dipole for which a statistical evaluation result is good (positive determination), and not adopting a dipole when no finding ids made as a result of a cross-sectional search (negative determination). A group of times at which the dipole estimation is performed in a cross-sectional manner is adopted as a dipole estimation time search range. The dipole estimation time search range is defined by a range that is detected from the range of the IED probability map, and therefore has a characteristic of being defined inward.

Meanwhile, it is basically possible to display the waveform additional information 9001 and the waveform additional information 9002 simultaneously in a superimposed manner.

An example of layers of waveform superimposed display in the display control unit 506 will be described below.

FIG. 10 is a diagram illustrating an example of the layers of the waveform superimposed display. The example illustrated in FIG. 10 is a configuration example of superimposed display of the waveform additional information illustrated in FIG. 9. The dipole estimation time search range is defined inside of the range of the IED probability map, and therefore can be represented by a display layer structure as illustrated in FIG. 10.

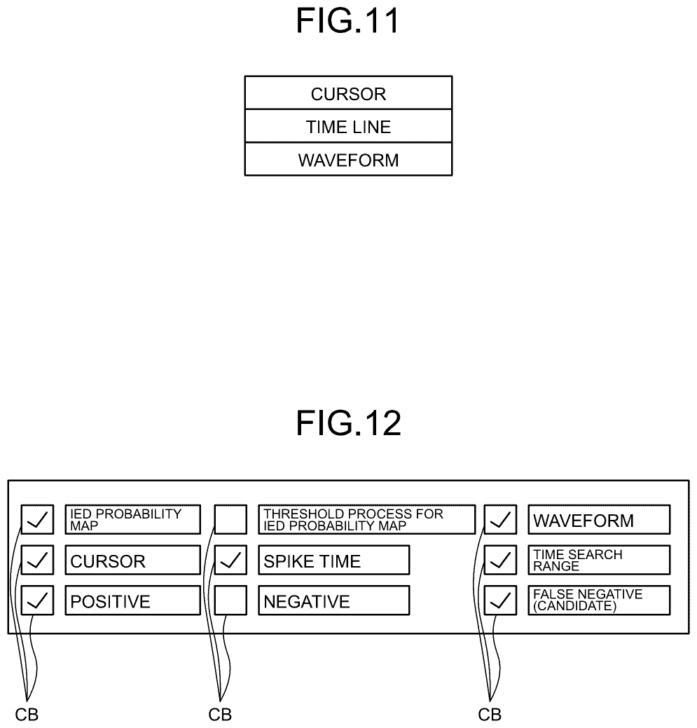

In the example illustrated in FIG. 10, the display control unit 506 draws and displays the IED probability drawing layer and a time search range layer in this order. Further, in the example illustrated in FIG. 11 (to be described later), the display control unit 506 displays the waveform, a time line, and the cursor illustrated in FIG. 9, in this order. Here, the three components, that is, the waveform, the time line, and the cursor illustrated in FIG. 9 are configured on two-dimensional arrangement as a drawing mode, and therefore can be easily distinguished from one another. Therefore, the three components need not always be displayed in this order. Further, if a cursor layer is not required as a display mode, the cursor layer may be omitted.

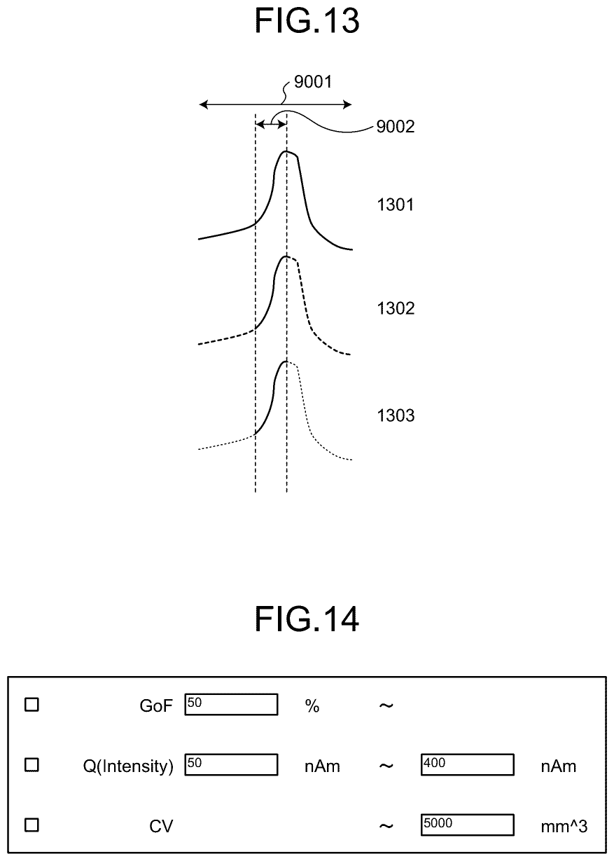

FIG. 11 is a diagram illustrating another example of the layers of the waveform superimposed display. The example illustrated in FIG. 11 is another mode of the configuration example of the superimposed display of the waveform additional information illustrated in FIG. 9. In the example illustrated in FIG. 11, an area image of the IED probability map drawing layer and the time search range layer is replaced with a waveform color. In the example illustrated in FIG. 11, for example, a reference display color of a waveform is determined as black, and a waveform color is set such that a color of a temporal range in which the IED probability value>0 is defined as a gradation between blue and red in accordance with an actual IED probability value. Further, in the example illustrated in FIG. 11, it may be possible to re-define and display the dipole estimation time search range in a color that does not belong to any of the colors as described above (see a reference symbol 1303 in FIG. 13 (to be described later)).

In addition, by setting a color of an interval corresponding to the waveform additional information 9002 illustrated in FIG. 9 to a complementary color of the color of the IED probability value, it is possible to represent the time search range without losing information on the probability value.

An example of selection of the layers to be displayed in the display control unit 506 will be described below.

FIG. 12 is a diagram illustrating an example of selection of layers to be displayed. The example illustrated in FIG. 12 is an example in which a user is allowed to select the layers to be displayed in accordance with need. When checkboxes CB illustrated in FIG. 12 are enabled, pieces of target data of the enabled checkboxes CB are displayed and unnecessary data can be hidden.

In the example illustrated in FIG. 10, display or hidden of each of the layers corresponds to the checkbox CB. In contrast, in the example illustrated in FIG. 11, regions of the IED probability map and the dipole estimation time search range are represented by the waveform colors, and, in this case, it is possible to express the same mode as hidden by returning the waveform colors to the reference color (black in this example).

The checkboxes CB for the positive, the negative, and the false negative candidate illustrated in FIG. 12 are examples of display of determination results. Meanwhile, the false positive candidate is included in the display of positive, and therefore, an independent checkbox CB is not prepared in the present embodiment; however, it may be possible to prepare the checkbox CB for the false positive candidate.

Another example of the waveform additional information that is collateral information of the waveform that is drawn by the collateral information drawing unit 507 in accordance with a predetermined time will be described below.

FIG. 13 is a diagram illustrating another example in which the waveform additional information is displayed in a superimposed manner. Similarly to the example illustrated in FIG. 9, the waveform additional information 9001 illustrated in FIG. 13 is an example of the display range of the IED probability map. The waveform additional information 9001 indicates a temporal range for providing a characteristic waveform. The waveform additional information 9002 is an example of display of the area corresponding to the time width that is retrieved as the candidate dipole estimation time in the display range of the IED probability map.

A reference symbol 1301 illustrated in FIG. 13 represents an example of normal waveform display. A dipole estimation time region is defined by adding vertical lines at boundaries corresponding to an upper limit time and a lower limit time of the waveform additional information 9002, and a range of a time estimation region is clarified by defining a waveform color in accordance with the probability value in the IED probability map of the waveform additional information 9001.

Meanwhile, according to the example illustrated in FIG. 13, it is advantageous in that only the color of the IED probability map can be drawn and visibility is improved; however, if the number of selected sensors increases, it becomes difficult to distinguish among boundary lines of the vertical lines, vertical lines that represent the dipole estimation times, and waveforms, which may lead to difficulty in identification of information.

A reference symbol 1302 illustrated in FIG. 13 represents a display example of dotted line display in which an interval that is retrieved by the dipole estimation is displayed by a solid line and a time range that includes the IED probability value in the search interval is displayed by a dotted line in the IED probability map. Meanwhile, the dotted line includes a style in which regions where dots are not drawn are displayed at predetermined transmittance such that a waveform line is not completely disconnected, and includes a combination of different expression methods, such as an interval between dots, a dotted line, a dashed line, a chain line, and a multi-dot chain line. In this manner, this expression method includes a change in an expression method for regions where dots are absent.

In the example illustrated in FIG. 13, it is not required to distinguish between the dipole estimation time search range and the IED probability map by colors in each of the cases illustrated in FIG. 10 and FIG. 11, and therefore, it is satisfactory to define the color of the waveform of the corresponding region in accordance with the IED probability value.

The reference symbol 1303 illustrated in FIG. 13 represents a display example in which a layer of the IED probability map and the dipole estimation time search range are displayed in different colors or with different shading. In this display example, it is disadvantageous in that IED probability map values are lost in the range of the dipole estimation time, but it is possible to improve visibility.

Meanwhile, to strictly hide the display, time and effort for changing the layer display are increased; however, in a practical sense, an IED probability map value that is displayed at a near time indicates an approximate value, so that a phase comes in which a focus is put on the matter of selection of a time at which the dipole estimation is to be performed after detection of the waveform. In this regard, if the estimation time search range is clarified, a user is able to practically use the search range without inconvenience in the determination. Further, with the method for the waveform additional information 9002, it is possible to visualize both without losing a numerical difference of the probability map values.

A method of determining suspicion of false negative in the threshold processing unit 503 will be described below.

The threshold processing unit 503 extracts sensors that have values equal to or larger than a predetermined probability at the detected peak time, as sensors that are used for dipole estimation. At this time, it is known that stability of a solution of the dipole estimation decreases with a decrease in the number of the sensors used for the dipole estimation. Therefore, in this example, if the number of the selected sensors does not reach a predetermined value, it may be possible not to perform the dipole estimation at the subject IED time. In this example, as a premise, it is assumed that the dipole estimation is not performed when the number of the sensors does not reach the predetermined value as described above.

The sensors are extracted when the number of the sensors that have the values equal to or larger than the predetermined probability obtained by the threshold processing unit 503 is equal to or larger than the predetermined value; however, if the number of the sensors does not reach the predetermined number, the dipole estimation is not performed and it is determined as negative. At around a boundary value of the predetermined value, there is a risk of false negative such that even when the IED probability is high, the number of the sensors does not reach the predetermined number and it is determined as negative. Therefore, when the number of the sensors does not reach the predetermined value, a predetermined criterion is set and an annotation label of false negative (candidate) is added to the subject time to clarify a display target.

For example, annotation of false negative (candidate) is added when the number of sensors is equal to or larger than X % (for example, 90%) of the predetermined value and Y % (for example, 75%) or more of the sensors have the IED probability map of Z % (for example, 90%) or more. With this configuration, it is possible to display one that may be false negative on a waveform screen, and a user is able to determine whether or not to adopt the one while understanding the risk.

Meanwhile, as another embodiment, it may be possible to adopt a part as a condition such that only X % or more of the predetermined value is simply adopted.

Furthermore, as another method of determining suspicion of false negative in the threshold processing unit 503, it may be possible to add the annotation of false negative (candidate) when the number of the sensors exceeds the predetermined value of sensor enhancement is performed with respect to the sensors that are calculated and adopted based on the threshold of the IED probability map.

For example, when the sensors that are selected in the IED probability map are overlooked in a three-dimensional space, in practical operation, spatially isolated points may be present in some cases. When the isolated points are regarded as a continuous region, it becomes fully possible to perform the dipole estimation when the number of sensors exceeds the predetermined value, and the isolated points may be determined as positive, and therefore, may be extract as false negative (candidate). As described above, it may be possible to further combine the methods of setting thresholds for Y and Z.

A method of determining suspicion of false positive in the threshold processing unit 503 will be described below.

The threshold processing unit 503 performs determination inversely against false negative, as an example of determination of suspicious of false positive. When a distribution of the IED probability values of the selected sensors are concentrated around the threshold, the threshold processing unit 503 may make extraction as suspicious of false positive and add annotation. For example, it may be possible to adopt a method of statistically evaluating an IED probability distribution such that a quartile range between the threshold and an upper limit value of the IED probability values adopted by the threshold processing unit 503 is calculated and if the whole or a predetermined percentage or more of the IED probability distribution is equal to or smaller than a first quartile point, it is determined as false positive.

Meanwhile, as another example of the determination of suspicious of false positive, it may be possible to adopt a method of determining as false positive based on statistical evaluation of the dipole estimation result.

An example of a setting scree of a dipole extraction condition in the threshold processing unit 503 will be described below.

FIG. 14 is a diagram illustrating an example of the setting screen of the dipole extraction condition. Among dipole estimation results that are registered as positive determination, as illustrated in FIG. 14 for example, references of Goodness of Fit (GoF), moment (Intensity), and Confidence Volume (CV) may be set as false positive determination thresholds, and annotation of false positive may be added when the thresholds are met. It may be possible to set the similar values in the statistical evaluation on the dipole estimation result in the waveform additional information 9002; however, in this example, the thresholds can be set separately from the statistical evaluation. If there is no difference, false positive does not occur, and if the difference occurs, distinguishing as false positive is possible. Further, application to an example the whole or part of the statistical evaluation on the dipole estimation is not performed is possible.

Meanwhile, the method of determining suspicious of false positive as described above may be combined with a certain condition, or may allow a user to select a threshold or a condition to be applied as illustrated in FIG. 14.

Furthermore, as still another example of the determination of suspicious of false positive, when sensors with high IED probabilities are separately distributed around a left temporal lobe and around a right temporal lobe (for example, a total of 60 sensors are selected such that a=30 on the left side and b=30 on the right side and a conditional parameter is set such that D=0.6>a/b>e=0.4), it may be possible to register a typical pattern indicating a measured waveform derived from a heart as a false positive pattern even if the number of sensors exceeds the predetermined value, and extract a case in which the condition is met as the false positive candidate.

A practical example of display by the display control unit 506 will be described below.

FIG. 15 is a diagram illustrating an example of display for correcting a result. The display illustrated in FIG. 15 represents a practical example of display including FIG. 7, FIG. 8, and FIG. 12.

A Contour Map 1501 illustrated in FIG. 15 is display in which a bird view of sensors that are present in a three-dimensional space is projected two-dimensionally, and magnetic field values of the respective sensors are displayed by shading of contours based on the magnetic field values. A step of (shading) below the Contour Map 1501 defines colors (shading) corresponding to a source to a sink of the magnetic field.

White circular marks in the Contour Map 1501 represent sensors and black circular marks represent displayed waveforms. Label names assigned to black circles in the Contour Map 1501 represent corresponding sensor labels.

The Contour Map 1501 allows selection of a sensor by drag operation by a mouse that is the input device 106 on the Contour Map 1501. Further, the Contour Map 1501 allows addition of a sensor by drag operation by the mouse with pressing of a predetermined key (shift key) of a keyboard that is the input device 106, and allows release of selection of a sensor by drag of the mouse with pressing of a control key.

Meanwhile, the example has been described above in which the spatial distribution of the sensors and the distribution based on the magnetic field values are displayed, but embodiments are not limited to this example. For example, when the technique is applied to electro-encephalography or a different waveform display system, it is possible to implement the same functions by displaying a different display means for visualizing a small waveform in a predetermined range before and after a cursor time for each of the sensors, so as to correspond to the subject sensor, rather than displaying shading of contours. For example, it may be possible to adopt a method of drawing the small waveform itself in a predetermined rectangular range of the subject sensor. Further, as another embodiment, it may be possible to adopt a method of representing a discrete representative value while adopting a sensor value as a color of the rectangle, instead of the contour.

A Sensor Select 1502 illustrated in FIG. 15 is for selecting and releasing a specific set of sensors at once by an arranged button. When a “select AI channel” button in the Sensor Select 1502 is pressed, a sensor with annotation that is closest to focused annotation (an arrow line in the upper part of the waveform) or a currently designated cursor line is selected. In other words, the “select AI channel” button implements a function to select a sensor that has been selected at the time of dipole estimation at a target time.

“Select all” in the Sensor Select 1502 allows selection of all of the sensors, and “release selection” in the Sensor Select 1502 changes states of all of the sensors to unselected states.

“LT (Left Temporal), LF (Left Frontal), LP (Left Parietal), or LO (Left Occipital)” in the Sensor Select 1502 indicates selection of all of the sensors located close to each of left brain areas. Further, “RT, RF, RP, or RO” in the Sensor Select 1502 selects a group of sensors corresponding to each of right brain areas. “ZT, ZC, ZP, or ZO” in the Sensor Select 1502 selects a group of sensors that are located at a center of separation. “L ALL” in the Sensor Select 1502 selects all of (LT, LF, LP, LO) simultaneously, and “R ALL” selects all of (RT, RF, RP, RO) simultaneously.

A Display Mode 1503 illustrated in FIG. 15 includes checkboxes S for selecting the display layers as illustrated in FIG. 12.

A Move Result 1504 illustrated in FIG. 15 is for moving the cursor to a temporal position at which annotation indicated by an arrow is added. In this case, a sensor is selected by selecting a waveform related to the subject annotation. However, it is not always required to adopt an implementation mode in which selection is forced, but it may be possible to adopt a mode in which only the cursor is moved while maintaining a previous selection state. In this case, for example, operation may be performed such that when the “select AI channel” button is pressed after the cursor is moved, a sensor of interest of the annotation is selected. The sensor of interest indicates a sensor that is used for calculation when the dipole estimation is performed.

A “Next” button in the Move Result 1504 moves the cursor to next annotation. A “Prev” button in the Move Result 1504 has a function to move the cursor to previous annotation. When next annotation is not present, it may be possible to disable the moving function or notify a user of the absence of the next annotation by displaying a message dialogue.

Meanwhile, as another embodiment, it may be possible to separately provide a move button with a focus on specific annotation, such as positive, false negative, or false positive. With this configuration, the user is allowed for transition.

The Annotation 1505 illustrated in FIG. 15 allows addition of a label to a temporal position that is designated by the cursor. By adding corresponding annotation to an annotation time of false positive, it is possible to reflect opinions of the user with respect to a positive result that is provided by the system, and give correction feedback information.

Define 1 (Negative) in the Annotation 1505 is an example of setting that is defined by the user, and it is assumed that the user is allowed to set an arbitrary name other than the specific annotation label described in this example.

Dipole Estimation 1506 illustrated in FIG. 15 has a function to perform dipole estimation. When an “Analysis” button in the Dipole Estimation 1506 is pressed, dipole estimation is performed by using the time designated by the cursor and waveform data of the currently selected sensor. An estimation result is drawn as an object that is formed of a sphere and a segment on a coronal cross-section 1508 and a Sagittal cross-section 1509 where the coordinates and the orientation are on an Axial cross-section 1507 on an MRI image, and numerical statistical information is displayed in value information 1510 of the dipole estimation result.

When a “Modify” button in the Dipole Estimation 1506 is pressed, and if a dipole analysis result of the subject annotation is registered, the dipole analysis result is replaced as an estimation result of the user analysis in the value information 1510 of the dipole estimation result. As an implementation mode of an effective range of the Modify function, it is sufficient to enable a process of the Modify button only when the cursor is included in a continuous region corresponding to annotation of interest (a continuous temporal region in which the probability map of each of the selected sensors used for the dipole estimation has a value larger than zero is calculated and a widest time range is adopted as the continuous region) and when the dipole estimation Analysis is performed in the continuous region.

“Add” in the Dipole Estimation 1506 allows addition of the dipole estimation result subjected to “Analysis” at the time indicated by the designated cursor position as annotation. When the annotation is already present, the dipole estimation result is registered in the subject annotation, and when annotation is not present, the dipole estimation result is registered as new annotation. Each of dipoles “Added” by the user can be subsequently used as positive annotation.

“Delete” in the Dipole Estimation 1506 is for deleting the dipole estimation result. When positive annotation has been added, the positive annotation is corrected to negative annotation. Alternatively, the user may add annotation again.

The reference symbol 1507 represents the Axial cross-section on the MRI image. In MRI, it is possible to simultaneously display a result of the value information 1510 of the dipole estimation result and dipole estimation corresponding to the user analysis, in a distinguishable manner. For example, colors of dipole drawing may be changed.

The reference symbol 1508 represents the Coronal cross-section on the MRI image. The reference symbol 1509 indicates the Sagittal cross-section on the MRI image.

The reference symbol 1510 indicates the value information on the dipole estimation. Time indicates a time at which the dipole estimation is performed, GoF indicates Good of Fitness, CV indicates Confidence Volume, X, Y, and Z indicate dipole estimation coordinates on MRI, Zdir and Xdir indicate dipole estimation directions on MRI, Q indicates moment (Intensity), and a result indicates a result of corresponding annotation. Meanwhile, Zdir indicates an angle θ between the dipole estimation direction and the Z axis on MRI, and Xdir indicates an angle φ between the dipole estimation direction and the Z axis on MRI.

Further, a result in the reference symbol 1510 indicates a result of the corresponding annotation. User analysis in the reference symbol 1510 displays a dipole estimation result at the time the “Analysis” button in the Dipole Estimation 1506 is pressed.

A specific example of correction in the collateral information drawing unit 507 will be described below.

On the waveform illustrated in FIG. 15, two kinds of annotation are added, such as annotation of the false negative candidate to which a dotted arrow and an exclamation mark are added and positive annotation to which an arrow is added.

A determination condition for false negative is set such that the number of selected sensors is equal to or larger than 90% of a predetermined value (50). In other words, the condition indicates that the number of sensors for which the IED probability values are equal to or larger than the threshold is equal to or larger than 45 and smaller than 50. In this example, the number of sensors for which the IED probability values are equal to or larger than the threshold is 47, and therefore, extraction as the false negative candidate is made.

The cursor is set at a time of the positive annotation. In a Display mode, as illustrated in the screen, an IED probability value, a waveform, a cursor, a spike time (dipole estimation time), and a time search range are enabled, and positive and the false negative candidate are enabled as for annotation.

### Check and Correction of Positive Annotation

Check and correction of positive annotation will be described below.

A user determines whether the annotation, such as the measured value visualized in the Contour Map 1501, the selected sensor, coordinates of dipole estimation, or an estimated time, is appropriate. If the annotation is not appropriate, the annotation is corrected.

The user moves the cursor line in a range in which the IED probability value is larger than zero, and selects an appropriate time while checking the Contour Map 1501.

The estimated time range indicates a range that is retrieved by the system, and therefore, the user determines whether it is better to perform correction in the retrieved range or it is better to deviate the retrieved range. When the user wants to select a more preferable sensor, the user selects a sensor again on the Contour Map 1501. Further, the user performs Analysis in the Dipole Estimation 1506 based on a combination of the designated cursor time and the re-selected sensor.

An execution result of the Analysis in the Dipole Estimation 1506 is displayed, as a dipole estimation result, in the user analysis 1510, the Axial cross-section 1507 on the MRI image, the Coronal cross-section 1508 on the MRI image, or the Sagittal cross-section 1509 on the MRI image.

If it is required to correct to a more preferable result from the Result, the user executes “Modify” in the Dipole Estimation 1506, so that the dipole estimation time and the selected sensor are overwritten with corrected annotation. In other words, although it is positive, it is determined that the time and the selected sensor of the annotation are not appropriate and the details are corrected.

Further, when the user determines, as a determination result of the user, that the annotation itself is negative, and if the user designates appropriate negative annotation from an annotation list in the Annotation 1505, the annotation is overwritten with corrected annotation. With this configuration, it is possible to reflect the determination made by the user in a final result, and it is possible to use the determination as feedback information to improve accuracy of the probability map.

### Check and Correction of False Negative Annotation

Check and correction of false negative annotation will be described below.

In the state as illustrated in FIG. 15, when the user selects Prev in the Move Result 1504, the cursor moves to a time that is indicated by an arrow of false negative annotation. Meanwhile, as the time of the arrow of the annotation for which a dipole estimation time is not registered as in the present embodiment, a time that is determined at the time of the threshold process is adopted.

A time of a selected subject 47 is automatically selected. Alternatively, the user may perform re-selection by executing “select AI channel” in the Sensor Select 1502.

With respect to annotation of the false negative candidate, it is not required to perform any operation when the user determines as negative as set, but it is possible to obtain more appropriate feedback by selecting appropriate Negative annotation in the Annotation 1505. When the user determines as positive, similarly to check of “positive annotation”, a sensor selection and a time are selected, Add in the Dipole Estimation 1506 is executed at a time at which an appropriate dipole estimation result is obtained by performing dipole estimation in the Dipole Estimation 1506, and a dipole estimation result is newly registered. The false negative annotation is corrected to positive annotation at the registered time. Meanwhile, the user may perform registration by Positive annotation in the Annotation 1505.

Thereafter, Next in the Move Result 1504 is performed, and annotation is sequentially corrected, so that confirmation of the result by the user is completed.

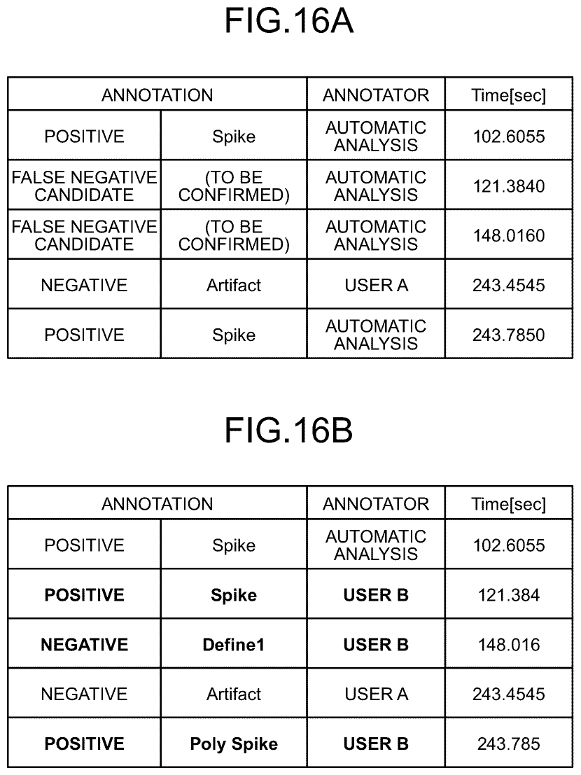

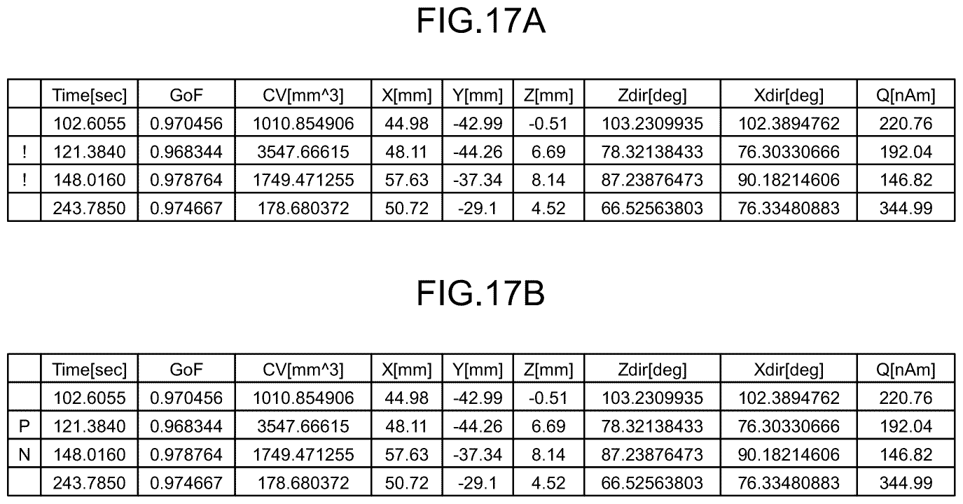

Further, as another embodiment, it may be possible to layout, as a part of a screen structure, a display system in which summarized annotation or a dipole indicating a positive result are displayed as a list as illustrated in FIGS. 16A and 16B and FIGS. 17A and 17B as will be described below, or it may be possible to implement a UI that operates in cooperative manner in a different window. Furthermore, the displayed lists may be combined and connected into a single table. In this case, if the dipole estimation result is absent, the dipole information may be left blank.

Moreover, it may be possible to adopt a mechanism in which only a specific classification list is displayed in conjunction with a selection state of the Display Mode 1503, so that it is possible to more efficiently reach target annotation and allow checking and correction in a concentrated manner.

A display example of annotation in the display control unit 506 will be described below.

FIGS. 16A and 16B are diagrams illustrating examples of display of annotation. The example illustrated in FIG. 16A is a display example that is obtained when a user A who has measured magneto-encephalography data adds, at the time of recording at 180 sec, annotation of Artifact to analysis data that is designated by the user, and thereafter performs the analysis flow as illustrated in FIG. 5 on the recorded data.

The example illustrated in FIG. 16B is a display example in which a user B subsequently performs correction after viewing the result illustrated in FIG. 16A.

### Description of Each Cell Item

As for annotation, it is possible to separately set information that indicates any of the positive, the false negative candidate, and the negative and an annotation name that is defined by the Annotation 1505. It is assumed that the positive is a label for an epileptic abnormal wave, the false negative candidate is a label for a candidate that is extracted by the system, and the negative is a label for other than directly epileptic abnormal wave, such as noise derived from metal or noise including a heart rate derived from a living body.

Further, the annotation may be changed by the user. The annotator is set by the user who has actually added the annotation, but the user of the annotation that is added by the analysis flow illustrated in FIG. 5 is represented as an automatic analysis. Setting can be made in accordance with the system such that a user name of the system (for example, a user name who has logged in the system) is set. The time indicates a time at which the annotation is added.

### Description Related Change in FIG. 16B

121.384 sec: an example in which the user B corrects the false negative candidate to positive and Spike
148.016 sec: an example in which the user B corrects the false negative candidate to negative and Define 1
243.785 sec: an example in which the user B subdivides Spike annotation into Poly Spike with respect to an automatic analysis result

In the present embodiment, the example is adopted in which only Spike annotation is added to all of positive by the automatic analysis, and a doctor further subdivides the annotation; for example, the doctor is allowed to subdivide the classification if required even when a large category is correct.

In addition, when the doctor additionally performs dipole analysis at a time at which analysis is not performed in the analysis flow illustrated in FIG. 5, it is possible to add a result to FIG. 16A. It is sufficient to adopt a time and an annotator that are designated by the user and set the annotation to an initial value that is determined in advance (for example, the annotation is set to positive and Spike or the annotation is unset).

By recording an edit result as described above, it is possible to allow a different user to verify the result and allow an analysis program to perform training.

Further, a display time that corresponds to details of the annotation is displayed. For example, by selecting a designated cell by click, the user is able to move to a target annotation time in FIG. 15 included in a row of the selected cell. It may be possible to adopt a method in which, by selecting a cell of the annotation, the user is directly select and edit the annotation. When the annotation is corrected, results in the main display and the waveform display in FIGS. 17A and 17B are corrected in a synchronized manner.

Like “(confirmation is required)” in the annotation, even in the annotation display, it is possible to display a predetermined identification result in an easily understandable manner to the user by adopting highlighted display.

FIG. 16A illustrates display that is displayed after the analysis flow illustrated in FIG. 5 is performed on the analysis data that is designated by the user.

A display example of a dipole list in the display control unit 506 will be described below.

FIGS. 17A and 17B are diagrams illustrating examples of display of the dipole list. The example illustrated in FIGS. 17A and 17B is an example in which a dipole estimation result for the positive annotation is displayed.

For example, by selecting a cell and selecting a dipole corresponding to a row of the cell, it is possible to move to a target annotation time as illustrated in FIG. 15.

When a dipole is corrected, results in the main display are also corrected in a synchronized manner. “!” is an example of highlighted display of the false positive candidate among positive. Even in the display of the dipole list, it is possible to provide the display in an easily understandable manner to the user by displaying a predetermined identification result in a highlighted manner.

With the mechanism as described above, it is possible to promptly access a point to be paid attention to by the user, check a result, and add or correct the dipole estimation result or the annotation label if required. Further, it is possible to store corrected result data, and gives, as feedback, information on improvement in performance of the system that outputs a probability map as systematized data.

The examples illustrated in FIGS. 17A and 17B correspond to FIG. 16A and FIG. 16B. FIG. 17A corresponds to FIG. 16A, and FIG. 17B corresponds to FIG. 16B.
121.384 sec: the example in which the user B corrects the false negative candidate to positive and Spike, where “!” is changed to “P” (Positive) display.
148.016 sec: the example in which the user B corrects the false negative candidate to negative and Define 1, where “!” is changed to “N” (Positive) display.
243.785 sec: the example in which the user B subdivides Spike annotation into Poly Spike with respect to the automatic analysis result; in this example, nothing is displayed because positive or negative is not changed and the result is only subdivided.

Meanwhile, in the example as described above, the highlighted display of “!” for the false negative candidate is changed to display for clarifying P or N to clarify that the user has edited, but it may be possible to adopt a different mode.

A modification of the annotation and the dipole list in the display control unit 506 will be described below.

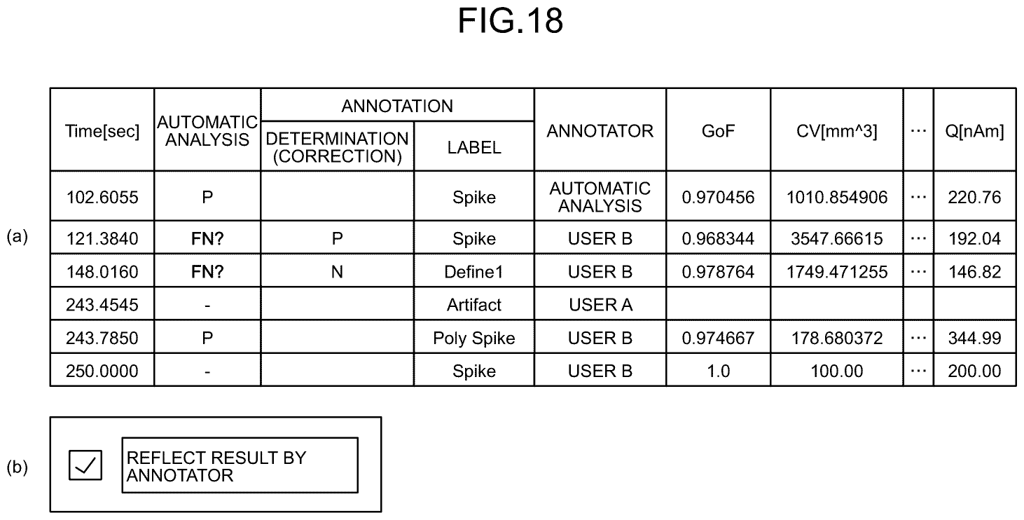

FIG. 18 is a diagram illustrating a modification of the annotation and the dipole list. The modification illustrated in FIG. 18 is an example in which the annotation and the dipole list are combined.

As illustrated at (a) in FIG. 18, a line of an automatic analysis is independently prepared, and positive (P), false negative candidate (FN?), and non-implementation of automatic analysis (-) are displayed. The annotation includes “determination (correction)” that reflects a determination result by the user and a “label”.

By adopting the format as described above, it is possible to recognize a state whether the automatic analysis is performed and whether the annotator has performed correction or addition. Further, as illustrated at (b) in FIG. 18, a checkbox of “reflect result of annotator” is prepared, and when the check is enabled, it is allowed to change display between positive and negative while a corrected portion of the annotator is reflected in the result of the automatic analysis. With this configuration, the user is able to examine the result while sequentially changing the result obtained by AI and the result provided by the annotator.

The flow of a series of operation will be described below.

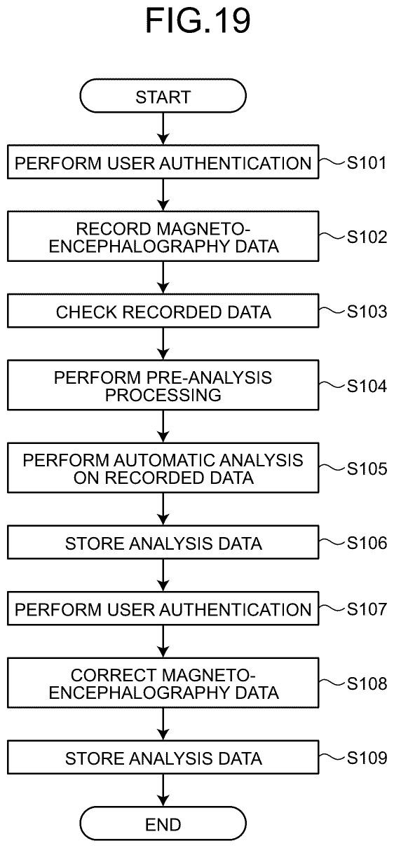

FIG. 19 is a flowchart illustrating the flow of a series of operation. As illustrated in FIG. 19, first, a display device of the information processing apparatus 50 has a scheme for performing login authentication in accordance with a user of the apparatus, and authentication is required before activation and use of the biological signal measurement system 1 (Step S101). With this configuration, when the user logs into the biological signal measurement system 1 at the time of activation, the user is allowed to access the biological signal measurement system 1.

Subsequently, the biological signal measurement system 1 measures a biological signal (Step S102). In the present embodiment, magneto-encephalography data is recorded as the biological signal.

Then, the information processing apparatus 50 displays pieces of waveform data of the magneto-encephalography signals obtained from the plurality of magnetic sensors and pieces of waveform data of the electro-encephalography signals obtained from the plurality of electrodes on the same time axis in a synchronized manner. Accordingly, the user is able to check the collected data. Further, the information processing apparatus 50 is able to add annotation at a designated time on the time axis as in the Contour Map 1501 (Step S103). For example, the user A is able to add Artifact to a disturbance noise waveform with respect to 243.4545 sec in FIGS. 16A and 16B.

Meanwhile, at Step S103, it is assumed that the user visually checks the waveform and adds the annotation in a state in which the waveform display is updated in real time while the waveform is being recorded (online display), but it may be possible to omit the mechanism as described above.

Subsequently, the information processing apparatus 50 performs pre-analysis processing (Step S104). The pre-analysis processing makes it possible to subsequently perform an analysis process by, as described in Japanese Unexamined Patent Application Publication No. 2006-167350, performing an image superimposition process for associating MRI and magneto-encephalography measurement data and thereafter setting a conductor model (sphere model) while viewing the MRI as illustrated in in FIG. 4 of Japanese Unexamined Patent Application Publication No. 2023-020297.

Meanwhile, when MRI is not provided, it may be possible to omit the image superimposition process, and set a sphere model by displaying sensor coordinates or marker coil coordinates in a spatial coordinate system of magneto-encephalography measurement instead of displaying the MRI as illustrated in FIG. 4 of Japanese Unexamined Patent Application Publication No. 2023-020297, or it may be possible to adopt automatic control for calculating predetermined coordinates and a radius from the sensor coordinates or the marker coil coordinates.

Subsequently, the information processing apparatus 50 performs automatic analysis on the recorded data (Step S105). The user at the time of the automatic analysis corresponds to “automatic analysis” in the example illustrated in FIGS. 16A and 16B.

Then, the information processing apparatus 50 stores automatic analysis data (Step S106).

Subsequently, the information processing apparatus 50 performs user authentication (Step S107). In this example, it is assumed that the operation is once completed at Step S106; however, the user whose login has been authenticated at Step S101 can continuously perform analysis at Step S108 (to be described later). In this case, user authentication at Step S107 may be omitted appropriately.

Then, the user who has performed the user authentication at Step S107 checks and corrects the magneto-encephalography data of the annotation in the display as illustrated in FIG. 15 to FIG. 18 (Step S108).

Lastly, the information processing apparatus 50 stores the analysis data that is checked and corrected at Step S108 in the same manner as Step S106 (Step S109).

In this manner, according to the embodiment, it is possible to visually display required information by displaying collateral information corresponding to a representative time of a determined waveform in accordance with a waveform classification result, and it is possible to provide information that allows a user for easy recognition. For example, it is possible to visually display information that is required to examine a positive result of an epileptic spike candidate, and it is possible to provide information that allows the user to easily recognize false negative.

Furthermore, the user is able to easily examine a determination result on positive or negative of the extracted characteristic waveform.

### Second Embodiment

A second embodiment will be described below.

The second embodiment is different from the first embodiment in that machine learning is used for generation of an IED probability map. In the description of the second embodiment below, explanation of the same components as the first embodiment will be omitted, and differences from the first embodiment will be described.

As for calculation of the IED probability map, it may be possible to adopt a related method using an algorithm for detecting a spike position (see A. Ossadtchi, S. Baillet, J. Mosher, D. Thyerlei, W. Sutherling, and R. Leahy, “Automated interictal spike detection and source localization in magneto-encephalography using independent components analysis and spatio-temporal clustering”, Clinical Neurophysiology, vol. 115, no. 3, pp. 508-522, 2004.), in addition to a method of application of a model that is calculated by using machine learning. When the machine learning is to be used, training is performed by adopting a time point of an IED that is manually detected by a doctor as a correct answer, and therefore, as compared to the related method of detecting a spike position, it is possible to obtain a result that is similar to a result obtained by analysis performed by the doctor. The related method is developed so as to detect an onset site of the IED or a peak of the IED; however, in actual analysis, a doctor performs analysis while adjusting a time at which dipole estimation is performed in a range from the onset site to the peak based on a waveform, diagnosis and opinion, or the like.

A case will be described below in which the IED probability map calculation unit 502 uses machine learning for generation of the IED probability map.

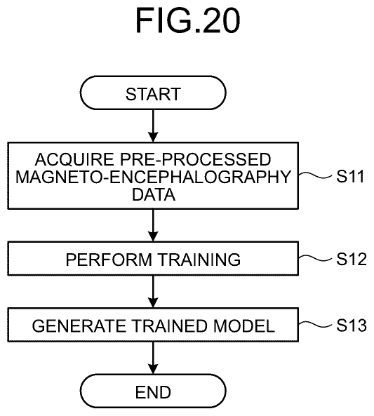

FIG. 20 is a flowchart illustrating the flow of a training process for generating an IED probability map by using machine learning according to the second embodiment. As illustrated in FIG. 20, the IED probability map calculation unit 502 acquires pre-processed magneto-encephalography data that is subjected to the pre-processing by the pre-processing unit 501 (Step S11), and performs training on the pre-processed magneto-encephalography data in order to output the IED probability map (Step S12). Meanwhile, as correct answer data that is used for the training, information on a sampling time point that is recorded when dipole estimation is performed and a sensor that is used for the dipole estimation is used.

Specifically, it is possible to apply a network that is frequently used in a task called Semantic Segmentation as represented by U-Net described in O. Ronneberger P. Fischer and T. Brox “U-net: Convolutional networks for biomedical image segmentation,” Proc. Int. Conf. Medical Image Comput. Comput.-Assisted Intervention, pp. 234-241 2015. Semantic Segmentation indicates a task for performing labeling on each of elements in all of arrays to be input, and, in a medical image, Semantic Segmentation is used when a region of a brain tumor or a cancer is automatically estimated. In this example, training is performed such that input magneto-encephalography data corresponds to mask data, where the mask data is generated from a sensor that is selected when the doctor performs dipole estimation and from an estimation time, while adopting the mask data as a correct answer. At this time, to improve generalization ability, it may be possible to adopt a data augmentation method, such as rearrangement of sensors in a random order or setting of a value in a certain range of a specific sensor to zero.

Furthermore, it is possible to similarly adopt a network that is used in a different task, such as Object Detection or Instance Segmentation, other than Semantic Segmentation. Object Detection is a method of estimating, by a rectangle, a position at which a target object is present in input arrays, and when this method is to be used, it is impossible to calculate the IED probability map, but it is possible to directly acquire a time at which dipole estimation is performed from the detected rectangle (for example, a midpoint of the rectangle) and a sensor. Instance Segmentation is a task in which Semantic Segmentation and Object Detection are mixed, and it is possible to calculate a region of a detected Object, in addition to classification and the number of the detected Object. Instance Segmentation is able to calculate the IED probability map, and therefore, can be applied as it is.

Subsequently, after the training at Step S2 is completed, the IED probability map calculation unit 502 generates a trained model (Step S13). The generated trained model is used for inference of machine learning, that is, when an IED is to be detected from unknown data.

As described above, according to the present embodiment, it is possible to perform additional training on the automatic analysis that is checked by the doctor, and it is possible to perform additional training on the dipole estimation result on the time data that is reviewed by a specific user, so that it is possible to improve accuracy of the learning model.

Meanwhile, in each of the embodiments as described above, when at least any of the functional units in the biological signal measurement system 1 is implemented by execution of a program, the program is provided by being incorporated in a ROM or the like. Further, the program that is executed by the biological signal measurement system 1 according to the embodiments as described above may be provided by being recorded in a computer-readable recording medium, such as a CD-ROM, a flexible disk (FD), a Compact Disc Recordable (CD-R), or a Digital Versatile Disk (DVD) in a computer-installable or a computer-executable file format.

Furthermore, the program that is executed by the biological signal measurement system 1 according to each of the embodiments as described above may be stored in a computer that is connected to a network, such as the Internet, and may be provided by download via the network.

Moreover, the program that is executed by the biological signal measurement system 1 according to each of the embodiments as described above may be provided or distributed via a network, such as the Internet. Furthermore, the program that is executed by the biological signal measurement system 1 according to each of the embodiments as described above has a module structure including at least any of the functional units as described above, and as actual hardware, each of the functional units as described above is loaded and generated on a main storage device by causing a CPU to read and execute the program from the ROM or the like.

Modes of the present invention includes followings, for example.

<1> A waveform display apparatus including:
a collateral information drawing unit configured to draw collateral information on waveforms with respect to the waveforms in accordance with a predetermined time, the waveforms being acquired by a plurality of sensors and displayed on a screen;
a waveform extraction unit configured to extract a characteristic waveform from waveform data;
a representative time determination unit configured to determine a representative time of the characteristic waveform extracted by the waveform extraction unit; and
a waveform classification unit configured to classify the characteristic waveform extracted by the waveform extraction unit, wherein
the collateral information drawing unit is configured to display collateral information corresponding to the representative time of the characteristic waveform in accordance with a classification result by the waveform classification unit, the representative time being determined by the representative time determination unit.

<2> The waveform display apparatus according to <1>, wherein
the waveform extraction unit is configured to compare each piece of waveform data acquired by the plurality of sensors with at least one piece of characteristic waveform information,
the representative time determination unit is configured to:
   determine appearance accuracy of the characteristic waveform information in at least a fixed interval in the waveform data, based on a correlation ratio between a peak interval of the waveform data and the characteristic waveform information; and
   identify a time at which an interval matching the characteristic waveform information appears, and a corresponding sensor, based on the appearance accuracy, and
the waveform classification unit is configured to determine a class of the characteristic waveform in accordance with the appearance accuracy information corresponding to the time determined by the representative time determination unit and the corresponding sensor.

<3> The waveform display apparatus according to <2>, wherein the collateral information drawing unit is configured to draw a number of the corresponding sensor corresponding to the time determined as the collateral information.

<4> The waveform display apparatus according to <3>, wherein the collateral information drawing unit is configured to perform highlighted display in accordance with the number of the corresponding sensor corresponding to the time determined as the collateral information.

<5> The waveform display apparatus according to any one of <1> to <3>, wherein the collateral information drawing unit is able to selectively perform display or highlighted display in accordance with a waveform classification result by the waveform classification unit.

<6> The waveform display apparatus according to <5>, wherein a class determined by the waveform classification unit includes a positive, a negative, a false negative candidate, and a false positive candidate.

<7> The waveform display apparatus according to <2>, wherein
the representative time determination unit is configured to determine the time at which the interval appears and the corresponding sensor from a plurality of candidate times, and
the collateral information drawing unit is configured to display the collateral information to be displayed, while including, in the collateral information, appearance accuracies and the plurality of candidate times in a predetermined range determined by the representative time determination unit.

<8> The waveform display apparatus according to <7>, wherein the collateral information drawing unit is configured to display the collateral information to be displayed, in colors corresponding to probability values and times determined as the plurality of candidate times.

<9> The waveform display apparatus according to <8>, wherein the collateral information drawing unit is configured to adopt complementary colors of colors of the probability values, for the plurality of candidate times.

<10> The waveform display apparatus according to <7>, wherein the collateral information drawing unit is configured to display the collateral information to be displayed with line styles changed corresponding to the probability values and times determined as the plurality of candidate times.

<11> A waveform display system including:
the waveform display apparatus according to any one of <1> to <10>; and
the plurality of sensors.

<12> A waveform display method implemented by a waveform display apparatus configured to display waveforms acquired by a plurality of sensors on a screen, the waveform display method including:
drawing collateral information on the waveforms in accordance with a predetermined time;
extracting a characteristic waveform from waveform data;
determining a representative time of the characteristic waveform extracted at the extracting; and
classifying the characteristic waveform extracted at the extracting, wherein
at the drawing, the collateral information corresponding to the representative time of the characteristic waveform determined at the determining is displayed in accordance with a classification result obtained at the classifying.

<13> A program that cause a computer to function as:
a collateral information drawing unit configured to draw collateral information on waveforms with respect to the waveforms in accordance with a predetermined time, the waveforms being acquired by a plurality of sensors and displayed on a screen;
a waveform extraction unit configured to extract a characteristic waveform from waveform data;
a representative time determination unit configured to determine a representative time of the characteristic waveform extracted by the waveform extraction unit; and
a waveform classification unit configured to classify the characteristic waveform extracted by the waveform extraction unit, wherein
the collateral information drawing unit is configured to display collateral information corresponding to the representative time of the characteristic waveform in accordance with a classification result by the waveform classification unit, the representative time being determined by the representative time determination unit.

According to an embodiment, it is possible to display required information in a visible manner, and provide information that allows a user for easy recognition.

The above-described embodiments are illustrative and do not limit the present invention. Thus, numerous additional modifications and variations are possible in light of the above teachings. For example, at least one element of different illustrative and exemplary embodiments herein may be combined with each other or substituted for each other within the scope of this disclosure and appended claims. Further, features of components of the embodiments, such as the number, the position, and the shape are not limited the embodiments and thus may be preferably set. It is therefore to be understood that within the scope of the appended claims, the disclosure of the present invention may be practiced otherwise than as specifically described herein.

The method steps, processes, or operations described herein are not to be construed as necessarily requiring their performance in the particular order discussed or illustrated, unless specifically identified as an order of performance or clearly identified through the context. It is also to be understood that additional or alternative steps may be employed.

Further, any of the above-described apparatus, devices or units can be implemented as a hardware apparatus, such as a special-purpose circuit or device, or as a hardware/software combination, such as a processor executing a software program.

Further, as described above, any one of the above-described and other methods of the present invention may be embodied in the form of a computer program stored in any kind of storage medium. Examples of storage mediums include, but are not limited to, flexible disk, hard disk, optical discs, magneto-optical discs, magnetic tapes, nonvolatile memory, semiconductor memory, read-only-memory (ROM), etc.

Alternatively, any one of the above-described and other methods of the present invention may be implemented by an application specific integrated circuit (ASIC), a digital signal processor (DSP) or a field programmable gate array (FPGA), prepared by interconnecting an appropriate network of conventional component circuits or by a combination thereof with one or more conventional general purpose microprocessors or signal processors programmed accordingly.

Each of the functions of the described embodiments may be implemented by one or more processing circuits or circuitry. Processing circuitry includes a programmed processor, as a processor includes circuitry. A processing circuit also includes devices such as an application specific integrated circuit (ASIC), digital signal processor (DSP), field programmable gate array (FPGA) and conventional circuit components arranged to perform the recited functions.

What is claimed is:

1. A waveform display apparatus comprising:
a collateral information drawing unit configured to draw collateral information on waveforms with respect to the waveforms in accordance with a predetermined time, the waveforms being acquired by a plurality of sensors and displayed on a screen;
a waveform extraction unit configured to extract a characteristic waveform from waveform data;
a representative time determination unit configured to determine a representative time of the characteristic waveform extracted by the waveform extraction unit; and
a waveform classification unit configured to classify the characteristic waveform extracted by the waveform extraction unit, wherein
the collateral information drawing unit is configured to display collateral information corresponding to the representative time of the characteristic waveform in accordance with a classification result by the waveform classification unit, the representative time being determined by the representative time determination unit.

2. The waveform display apparatus according to claim 1, wherein
the waveform extraction unit is configured to compare each piece of waveform data acquired by the plurality of sensors with at least one piece of characteristic waveform information,
the representative time determination unit is configured to:
determine appearance accuracy of the characteristic waveform information in at least a fixed interval in the waveform data, based on a correlation ratio between a peak interval of the waveform data and the characteristic waveform information; and
identify a time at which an interval matching the characteristic waveform information appears, and a corresponding sensor, based on the appearance accuracy, and
the waveform classification unit is configured to determine a class of the characteristic waveform in accordance with the appearance accuracy information corresponding to the time determined by the representative time determination unit and the corresponding sensor.

3. The waveform display apparatus according to claim 2, wherein the collateral information drawing unit is configured to draw a number of the corresponding sensor corresponding to the time determined as the collateral information.

4. The waveform display apparatus according to claim 3, wherein the collateral information drawing unit is configured to perform highlighted display in accordance with the number of the corresponding sensor corresponding to the time determined as the collateral information.

5. The waveform display apparatus according to claim 1, wherein the collateral information drawing unit is able to selectively perform display or highlighted display in accordance with a waveform classification result by the waveform classification unit.

6. The waveform display apparatus according to claim 5, wherein a class determined by the waveform classification unit includes a positive, a negative, a false negative candidate, and a false positive candidate.

7. The waveform display apparatus according to claim 2, wherein
the representative time determination unit is configured to determine the time at which the interval appears and the corresponding sensor from a plurality of candidate times, and
the collateral information drawing unit is configured to display the collateral information to be displayed, while including, in the collateral information, appearance accuracies and the plurality of candidate times in a predetermined range determined by the representative time determination unit.

8. The waveform display apparatus according to claim 7, wherein the collateral information drawing unit is configured to display the collateral information to be displayed, in colors corresponding to probability values and times determined as the plurality of candidate times.

9. The waveform display apparatus according to claim 8, wherein the collateral information drawing unit is configured to adopt complementary colors of colors of the probability values, for the plurality of candidate times.

10. The waveform display apparatus according to claim 7, wherein the collateral information drawing unit is configured to display the collateral information to be displayed with line styles changed corresponding to the probability values and times determined as the plurality of candidate times.

11. A waveform display system comprising:
the waveform display apparatus according to claim 1; and
the plurality of sensors.

12. A waveform display method implemented by a waveform display apparatus configured to display waveforms acquired by a plurality of sensors on a screen, the waveform display method comprising:
drawing collateral information on the waveforms in accordance with a predetermined time;
extracting a characteristic waveform from waveform data;
determining a representative time of the characteristic waveform extracted at the extracting; and
classifying the characteristic waveform extracted at the extracting, wherein
at the drawing, the collateral information corresponding to the representative time of the characteristic waveform determined at the determining is displayed in accordance with a classification result obtained at the classifying.

13. A non-transitory computer-readable medium including programmed instructions that cause a computer to function as:
a collateral information drawing unit configured to draw collateral information on waveforms with respect to the waveforms in accordance with a predetermined time, the waveforms being acquired by a plurality of sensors and displayed on a screen;
a waveform extraction unit configured to extract a characteristic waveform from waveform data;
a representative time determination unit configured to determine a representative time of the characteristic waveform extracted by the waveform extraction unit; and
a waveform classification unit configured to classify the characteristic waveform extracted by the waveform extraction unit, wherein
the collateral information drawing unit is configured to display collateral information corresponding to the representative time of the characteristic waveform in accordance with a classification result by the waveform classification unit, the representative time being determined by the representative time determination unit.
